(19) United States
(12) Patent Application Publication
(10) Pub. No.: US 2017/0369570 A1
(43) Pub. Date: Dec. 28, 2017

(54) COMPOSITIONS AND METHODS FOR CANCER IMMUNOTHERAPY

(71) Applicant: IMMUNEXCITE, INC., Waltham, MA (US)

(72) Inventors: Ifat Rubin-Bejerano, Newton, MA (US); Gabriel Oscar Reznik, Bedford, MA (US); Zuzana Dostalova, Cambridge, MA (US); Isabelle Sansal-Castellano, Jamaica Plain, MA (US)

(21) Appl. No.: 15/543,366

(22) PCT Filed: Jan. 20, 2016
(86) PCT No.: PCT/US2016/014171

§ 371 Date: Jul. 13, 2017

Related U.S. Application Data
(60) Provisional application No. 62/105,683, filed on Jan. 20, 2015.

(51) Int. Cl. C07K 16/28; A61K 47/61; A61K 39/395; A61K 39/00

(52) U.S. Cl. CPC C07K 16/28; A61K 39/395; A61K 47/61; A61K 2039/505

(57) ABSTRACT

The present invention includes, among other things, β-1,6-glucan linked to an antibody directed to a cell present in a tumor microenvironment. T regulatory cells can be one type of cell present in the tumor microenvironment. In particular embodiments, the antibody is directed to a surface feature of a T regulatory cell, MDSC, or other immune cell. In certain embodiments, the antibody is directed to T regulatory cells, MDSCs, or other immune cells present in a tumor microenvironment. Compositions including β-1,6-glucan linked to an antibody directed to a T regulatory cell, MDSC, or other immune cell may be useful, e.g., in the treatment of a tumor or cancer in a subject in need thereof. Accordingly, the present invention provides, among other things, methods and compositions for treatment of a tumor or cancer.

### CROSS-REFERENCE TO RELATED APPLICATIONS

This application claims priority from U.S. Provisional Patent Application No. 62/105,683, filed Jan. 20, 2015, the disclosure of which is hereby incorporated by reference in its entirety.

### BACKGROUND

Many factors contribute to the development of a tumor or cancer. For instance, cancer cells can develop, or develop as the result of, insensitivity to growth-inhibitory signals, decreased requirement for growth signals, acceleration of the cell cycle, decreased contact inhibition, reduced apoptosis, decreased differentiation, changes in cell membrane composition, changes in organellar characteristics, or metabolic shifts. Other factors that can contribute to the development of cancer or tumor growth relate to the environment or microenvironment of cancer or tumor cells. Such environmental or microenvironmental factors can include, for example, secreted soluble factors, presence or absence of particular signaling molecules, factors relating to angiogenesis, pH, interstitial pressure, mechanical cues, changes in extracellular matrix, and the recruitment of cells of particular types, e.g., immune cells, endothelial cells, fibroblasts, myofibroblasts, and/or pericytes. Cancer remains a significant medical challenge. There is a need in the art for compositions and methods for the treatment of cancer.

### Definitions

The term “antigen”, as used herein, refers to an agent that elicits an immune response; and/or an agent that binds to a T cell receptor (e.g., when presented by an MHC molecule) or to an antibody. In some embodiments, an antigen elicits a humoral response (e.g., including production of antigen-specific antibodies); in some embodiments, it elicits a cellular response (e.g., involving T-cells whose receptors specifically interact with the antigen). In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer, e.g., other than a nucleic acid or amino acid polymer), etc. In some embodiments, an antigen is or comprises a polypeptide. In some embodiments, an antigen is or comprises a glycan, e.g., a glucan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source).

As used herein, “binding moiety” or “targeting moiety” means any molecule capable of selectively interacting with one or more antigen targets. For instance, a binding moiety of the present invention can be a peptide, polypeptide, antibody, antibody fragment, small molecule, nucleic acid, aptamer, or other molecule capable of selectively binding one or more antigen targets.

As used herein, “antibody” means an immunoglobulin molecule that recognizes and specifically binds to a target, such as a protein, polypeptide, peptide, carbohydrate, polynucleotide, lipid, or combination thereof through at least one antigen recognition site within a variable, optimized, or selected region of an immunoglobulin molecule. As used herein, the term “antibody” encompasses intact polyclonal antibodies, intact monoclonal antibodies, antibody fragments (such as Fab, Fab′, F(ab′)₂, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as glucans, toxins, radioisotopes, and the like. As used herein, an antibody can be, e.g., an “intact antibody” or an “antibody fragment.” As used herein, “antibody” additionally encompasses various alternative formats as may be known in the art, e.g., camelid antibodies.

As used herein, an antibody or intact antibody can be an immunoglobulin molecule comprising four polypeptide chains, two heavy (H) chains and two light (L) chains inter-connected by disulfide bonds. Each heavy chain comprises a heavy chain variable (V_{H}) region and a heavy chain constant region (C_{H}). The heavy chain constant region comprises three domains, C_{H}L C_{H}2 and C_{H}3. Each light chain comprises a light chain variable (V_{L}) region and a light chain constant region (C_{L}). The V_{H} and V_{L} regions can be further subdivided into regions of hypervariability, termed complementarity determining regions (CDR), interspersed with regions that are more conserved, termed framework regions (FR). Other intact antibodies, e.g., intact camelid antibodies, are known in the art.

As used herein, the term “antibody fragment” means a molecule comprising at least a portion of an immunoglobulin protein, such as, for example, the antigen-binding or variable region of an antibody. Examples of antibody fragments include Fab, Fab′, F(ab′)₂, and FIT fragments; triabodies; tetrabodies; linear antibodies; single-chain antibody molecules; and CDR-containing moieties included in multi-specific antibodies formed from antibody fragments. Those skilled in the art will appreciate that the term “antibody fragment” does not imply and is not restricted to any particular mode of generation. An antibody fragment may be produced through use of any appropriate methodology, including but not limited to cleavage of an intact antibody, chemical synthesis, recombinant production, etc.

The term “surface feature,” as used herein, means an antigen present on a cell such that the antigen is available for binding with one or more binding moieties. A surface feature may be, e.g., a protein, peptide, polypeptide, lipid, other molecule, or a combination thereof. A surface feature may be inclusive of all or a portion of a single protein, peptide, polypeptide, lipid, or other molecule, two or more proteins, peptides, polypeptides, lipids, or other molecules, or a combination of any of two or more cellular components selected from proteins, peptides, polypeptides, lipids, or other molecules. A cell may include a particular surface feature or group of surface features, e.g., continuously, transiently, discontinuously, or for any period of time, e.g., a period of time defined by a particular cellular state, phase, or condition or a stochastically defined period of time.

As used herein, the term “receptor” means a cell surface feature which is directed to a ligand whereby the binding of the ligand modulates the activity of the cell surface feature. In particular instances, the binding of a ligand by a receptor results in the transduction of a signal in that the receptor modulates a change in one or more downstream molecules in a signaling pathway. A receptor, like various other cellular structures or pathways, can be “agonized” in that it can be modulated in a manner that causes the receptor to increase in activity or “antagonized” in that it can be modulated in a manner that causes the receptor to decrease in activity.

As used herein, the term “immunoreceptor” means a receptor capable of modulating an immune activity of a cell. In various embodiments, an immunoreceptor may be a checkpoint, e.g., a checkpoint for an immune activity.

As used herein, the term “T regulatory cell function” means the ability to participate, or activate participation of, one or more T regulatory cells in a systemic or local mechanism that directly or indirectly reduces the net activity of any of one or more immune or immune-associated cell types or biological processes. In particular examples, “T regulatory cell function” can include any of one or more of T cell inhibition (e.g., inhibition of T cell induction and/or proliferation), inhibition of inflammation, and inhibition of self-antigen immunotargeting (e.g., prevention of an autoimmune response).

As used herein, the term “glucan-conjugated” or “glucan-linked” as well as grammatical equivalents thereof means that a molecule is stably associated with one or more glucan molecules. A molecule, e.g., a binding moiety, may be stably associated with a glucan, e.g., directly or indirectly, e.g., via a direct covalent bond or via a linker (including a non-covalently bound linker such as a biotin-avidin linker).

As used herein, the term “subject” means any mammal, including humans. In certain embodiments of the present invention the subject is an adult, an adolescent or an infant.

As used herein, the term “treatment” (also “treat” or “treating”), in its broadest sense, refers to any administration of a substance (e.g., provided compositions) that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be administered to a subject who does not exhibit signs of the relevant disease, disorder and/or condition and/or of a subject who exhibits only early signs of the disease, disorder, and/or condition. Alternatively or additionally, in some embodiments, treatment may be administered to a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein, the term “therapeutically effective amount” is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular subject. Rather, a therapeutically effective amount may be that amount that provides a particular desired pharmacological response in a significant number of subjects when administered to subjects in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

### BRIEF DESCRIPTION OF THE DRAWINGS

FIG. 1 is a chart showing ELISA results. FIG. 1 includes four columns showing data from conjugates (anti-CTLA-4 antibody conjugated to β-1,6-glucan) having different β-1,6-glucan loads and different levels of binding by anti-β-1,6-glucan antibodies.

FIG. 2A shows data from mice with tumors (volume range of 130-170 mm³), which mice were treated with 10 mg/kg of anti-CTLA-4 antibody or anti-CTLA-4 antibody conjugated to β-1,6-glucan (denoted “mAbXcite” in the figure), and were dosed with 100 μl of 10% IVIG. The mice were treated twice weekly for 2 weeks (40 mg/kg in 4 doses). Tumor volume was calculated as L×W×W/2, and the medians are shown.

FIG. 2B shows data from mice with tumors (volume range of 130-170 mm³), which mice were treated with 10 mg/kg of anti-CTLA-4 antibody or anti-CTLA-4 antibody conjugated to β-1,6-glucan (denoted “mAbXcite” in the figure), and were dosed with 30 purified anti-β-1,6-glucan IgGs. The mice were treated twice weekly for 2 weeks (40 mg/kg in 4 doses). Tumor volume was calculated as L×W×W/2, and the medians are shown.

FIG. 2C shows data from mice with tumors (volume range of 130-170 mm³), which mice were treated with 5 mg/kg of anti-CTLA-4 antibody or anti-CTLA-4 antibody conjugated to β-1,6-glucan (denoted “mAbXcite” in the figure), and were fed with bread. The mice were treated twice weekly for 3 weeks (30 mg/kg in 6 doses). Tumor volume was calculated as L×W×W/2, and the medians are shown.

FIG. 3A shows an image of a tumor sample that was fixed and stained. The sample was from a mouse fed a regular diet supplemented with bread, which mouse was dosed with PBS. Four hours following dosing the tumor was fixed. Neutrophil immunohistochemistry staining was performed by using the monoclonal antibody NIMP-R14, which is highly specific for the murine neutrophil marker Ly-6G and Ly-6C.

FIG. 3B shows an image of a tumor sample that was fixed and stained. The sample was from a mouse fed a regular diet supplemented with bread, which mouse was dosed with 10 mg/kg of anti-CTLA-4 antibody. Four hours following dosing the tumor was fixed. Neutrophil immunohistochemistry staining was performed by using the monoclonal antibody NIMP-R14, which is highly specific for the murine neutrophil marker Ly-6G and Ly-6C.

FIG. 3C shows an image of a tumor sample that was fixed and stained. The sample was from a mouse fed a regular diet supplemented with bread, which mouse was dosed with anti-CTLA-4 antibodies conjugated to β-1,6-glucan. Four hours following dosing the tumor was fixed. Neutrophil immunohistochemistry staining was performed by using the monoclonal antibody NIMP-R14, which is highly specific for the murine neutrophil marker Ly-6G and Ly-6C.

### DETAILED DESCRIPTION

Without wishing to be bound by any particular scientific theory, at least one theory relating to tumor development suggests that tumors develop through a process of immunoediting, such that the presence of a tumor is indicative of a failure by the host immune system to check tumor progression. This theory, in some embodiments, asserts that immunoediting includes three distinct phases: an elimination stage in which immune functions effectively suppress tumor development, an equilibrium stage in which aberrant (e.g., pre-tumor) cells survive but are held in check, and an escape stage in which one or more aberrant cells are able to develop into clinically recognizable tumors. Tumor microenvironments can be immunosuppressive. The contribution of stroma cells in the immune escape can be represented by a rapid recruitment, expansion, and/or activation of various immunosuppressive cells of lymphoid and myeloid origin in the tumor microenvironment, including, e.g., regulatory T cells (Tregs), tumor-associated M2 macrophages (TAMs), Tie2-expressing monocytes, N2 neutrophiles, regulatory/tolerogenic dendritic cells (DCs), and/or myeloid-derived suppressor cells (MDSCs).

A variety of mechanisms can contribute to the ability of tumors to grow in a host organism despite the presence of the organism's immune system. Aspects of the immune system can include, both systemically and in a tumor microenvironment, immune checkpoints and/or T regulatory cells (inclusive of T regulatory cells as a checkpoint and checkpoints present in or on T regulatory cells and/or T cells). Checkpoints in the immune system are thought to normally play a role in maintaining immune homeostasis. Immune checkpoints can be cell surface molecules that regulate immune response, e.g., limiting autoimmunity by mediating inhibitory signaling pathways. T regulatory cells are thought to normally regulate a variety of immune functions and in particular to contribute to the suppression of immune responses. Without wishing to be bound by any particular scientific theory, immunosuppressive activities of T regulatory cells are hypothesized to function in hosts under non-disease conditions to curtail T cell responses against self-antigens and allergens, thus preventing, e.g., autoimmune diseases and allergic reactions or allergies. Such checkpoint activity, however, can limit the immune response in certain conditions under which a more active immune response may cumulatively contribute to the wellness of a host and, in particular examples, may contribute to the development of a tumor or cancer. In certain diseases, such as cancer, T regulatory cells may inhibit immune responses that would otherwise inhibit the growth of tumor cells.

The present invention provides, among other things, binding moieties directed to a non-tumor cell present in a tumor microenvironment, the binding moiety including a β-1,6-glucan. Glucans are polysaccharides found in many species of lichenized fungi. β-1,6-glucan has been found to induce a potent anti-fungal response. The present inventors have found that β-1,6-glucan is capable of stimulating complement activation and is capable of mediating efficient phagocytosis.

The present invention includes, among other things, β-1,6-glucan linked to an antibody directed to a non-tumor cell present in a tumor microenvironment. A T regulatory cell is one type of cell that can be present in the tumor microenvironment. In particular embodiments, the antibody is directed to a surface feature of a T regulatory cell. In certain embodiments, the antibody is directed to T regulatory cells present in a tumor microenvironment. Compositions including β-1,6-glucan linked to an antibody directed to a T regulatory cell may be useful, e.g., in the treatment of a tumor or cancer in a subject in need thereof. Another type of cell that can be present in a tumor microenvironment is a T cell. In particular embodiments, the antibody is directed to a surface feature of a T cell. In certain embodiments, the antibody is directed to T cells present in a tumor microenvironment. Compositions including β-1,6-glucan linked to an antibody directed to a T cell may be useful, e.g., in the treatment of a tumor or cancer in a subject in need thereof. Other cells commonly present in the tumor microenvironment can include Myeloid Derived Suppressor Cells (MDSCs). In certain embodiments, antibody is directed to MDSCs present in a tumor microenvironment. Among other things, compositions including β-1,6-glucan linked to an antibody directed to MDSCs are contemplated. Further contemplated are other immune cells such as M2 macrophages and the like. The present invention provides, among other things, the use of β-1,6-glucan linked to an antibody directed to a non-tumor cell present in a tumor microenvironment to treat a subject in need thereof, e.g., a subject having a tumor.

### Binding Moieties

A binding moiety of the present invention can include any molecule capable of selectively interacting with one or more targets, e.g., a surface feature of a T regulatory cell in a tumor microenvironment. For instance, a binding moiety of the present invention can be a peptide, polypeptide, peptide or polypeptide which is directed to a transmembrane molecule (e.g., a natural or synthetic ligand), naturally occurring peptide ligand for a receptor or modified form thereof, engineered binding protein, antibody, antibody fragment, receptor, ligand, small molecule, nucleic acid, or aptamer. In particular embodiments, a binding moiety of the present invention is an antibody or antibody fragment. In various embodiments of the present invention, any binding moiety provided herein may be conjugated to a glucan, e.g., β-1,6-glucan.

In various embodiments of the present invention, a binding moiety of the present invention is directed to one or more non-tumor cells present in the tumor microenvironment. In various embodiments of the present invention, a binding moiety of the present invention is directed to T regulatory cells. In various embodiments of the present invention, a binding moiety of the present invention is directed to one or more surface features of a T regulatory cell, such as one or more surface features selectively present on T regulatory cells of a tumor host organism. In particular embodiments, a binding moiety of the present invention is directed to T regulatory cells present in a tumor microenvironment, e.g., T regulatory cells present in the tumor microenvironment of a tumor of a host organism. In various embodiments of the present invention, a binding moiety of the present invention is directed to MDSCs. In various embodiments of the present invention, a binding moiety of the present invention is directed to one or more surface features of a MDSC, such as one or more surface features selectively present on MDSCs of a tumor host organism. In particular embodiments, a binding moiety of the present invention is directed to MDSCs present in a tumor microenvironment, e.g., MDSCs present in the tumor microenvironment of a tumor of a host organism.

In various embodiments of the present invention, a β-1,6-glucan-linked binding moiety of the present invention is directed to one or more non-tumor cells present in the tumor microenvironment. In various embodiments of the present invention, a β-1,6-glucan-linked binding moiety of the present invention is bispecfic and is directed to one non-tumor cell and to one tumor cell. In various embodiments of the present invention, a β-1,6-glucan-linked binding moiety of the present invention is directed to T regulatory cells or MDSCs. In various embodiments of the present invention, a β-1,6-glucan-linked binding moiety of the present invention is directed to one or more surface features of T regulatory cells or MDSCs, such as one or more surface features selectively present on T regulatory cells or MDSCs of a tumor host organism. In particular embodiments, a β-1,6-glucan-linked binding moiety of the present invention is directed to T regulatory cells or MDSCs present in a tumor microenvironment, e.g., T regulatory cells or MDSCs present in the tumor microenvironment of a tumor of a host organism.

In certain embodiments, a binding moiety of the present invention is a binding moiety capable of selectively binding one or more receptors present on a T regulatory cell.

In various instances, a composition of the present invention may bind one or more receptors present on a T regulatory cell in a manner that decreases the activity of the T regulatory cell by agonizing an inhibitory receptor or receptor that, upon activation, contributes negatively to the activity of a T regulatory cell or decreases T regulatory cell activity.

In various instances, a composition of the present invention may bind one or more receptors present on a T regulatory cell in a manner that decreases the activity of the T regulatory cell by antagonizing a stimulatory receptor or receptor that, upon activation, contributes positively to the activity of a T regulatory cell or promotes T regulatory cell activity.

Antibodies that target immune checkpoints can antagonize inhibitory immunologic pathways or activate stimulatory immunologic pathways. In various examples of such binding moieties as provided herein, immune checkpoint targeted binding moieties are capable of eliciting an anti-tumor effect.

In particular instances, a binding moiety of the present invention may be directed to a surface feature of a T regulatory cell. Methods of identifying T regulatory cells, e.g., in vivo or ex vivo, are known in the art. Various T regulatory cells may express, e.g., one or more of CD4 (CD4+), Foxp3 (Foxp3+), IL-2R/CD25 (CD25+), CTLA-4 (CD152), glucocorticoid-induced TNF receptor (GITR), the high affinity IL-2 receptor α-chain, CXCR3, CXCR4, Neuropilin-1 (Nrp-1), Garpin (GARP; glycoprotein A repetitions predominant), lymphocyte activation gene-3 (LAG-3), TNFR-2, TIM-3, ICOS, CCR6, CCR8, CCR10, B7/CD28, B7/CD28-like targets, ILDR2 or a combination thereof, any, one or more, or all of which may be expressed, e.g., as a surface feature or as a component of a surface feature. Those of skill in the art will understand that this list is neither exhaustive of nor limiting to those proteins and/or surface features that may be expressed by or present on a T regulatory cell, and moreover that the inclusion of any of one or more proteins herein is not intended to exclude any of one or more cells or cell types that do not express the protein in general or any under particular condition.

T regulatory cells may include, without limitation, CD4+Foxp3+ cells, CD4+Foxp3+CD25+ cells, induced Treg cells (iTreg), natural Treg cells, adaptive Treg cells, thymus-derived CD4+Treg cells, extrathymically-derived CD4+Treg cells, Tr1-cells, and CD4+Foxp3− cells. Those of skill in the art will understand that this list is neither exhaustive of nor limiting to those proteins and/or surface features that may be expressed by or present on a T regulatory cell, and moreover that the inclusion of any of one or more proteins herein is not intended to exclude any of one or more cells or cell types that do not express the protein in general or any under particular condition.

In various embodiments, a binding moiety of the present invention binds a T regulatory cell surface feature that is or includes as a component of one or more of CD4 (CD4+), Foxp3 (Foxp3+), IL-2R/CD25 (CD25+), CTLA-4 (CD152), glucocorticoid-induced TNF receptor (GITR), the high affinity IL-2 receptor α-chain, CXCR3, CXCR4, Neuropilin-1 (Nrp-1), Garpin (GARP; glycoprotein A repetitions predominant), lymphocyte activation gene-3 (LAG-3), TNFR-2, TIM-3, ICOS, CCR6, CCR8, CCR10, programmed death-1 (PD-1), programmed death ligand-1 or -2 (PD-L1, PD-L2), lymphocyte activation gene-3, T cell immunoglobulin mucin protein-3, GITR, and CD-137.

Binding moieties of the present invention may be, without limitation, agonists or antagonists of immunoreceptors expressed by T regulatory cells. In embodiments in which a binding moiety is able to inhibit T regulatory cell activity, a binding moiety may be, e.g., a binding moiety that antagonizes a receptor that promotes T regulatory cell activity or a binding moiety that agonizes a receptor that inhibits T regulatory cell activity.

Without limitation, examples of a binding moiety that antagonizes a receptor that promotes T regulatory cell activity as described herein includes binding moieties that antagonize one or more of PD-1, CTLA-4, TIM-3, LAG-3, ICOS, or TIGIT. Particular examples of such binding moieties include, without limitation, the anti-PD1 antibody pembrolizumab, the anti-PD1 antibody nivolumab, and the anti-CTLA-4 antibody ipilimumab. Without limitation, examples of a binding moiety that agonizes a receptor that inhibits T regulatory cell activity as described herein include binding moieties that agonize GITR, OX-40 or CD40. Particular examples of such binding moieties include, without limitation, the anti-GITR antibody MK-4166.

### Glucans

A glucan of the present invention, e.g., a β-1,6-glucan, can be derived from or synthesized from any source and/or by any procedure, e.g., any source and/or by any procedure known in the art.

In one embodiment, the glucan is isolated or derived from a lichen, which in one embodiment is from the genus Umbilicariaceae. In one embodiment, the glucan is isolated from a fungus. In one embodiment, the glucan is isolated from yeast, or in another embodiment the glucan is chemically synthesized or acetylated. In another embodiment, the glucan is conjugated to a solid support.

Glucans are glucose-containing polysaccharides found inter alia in fungal cell walls. α-glucans include one or more α-linkages between glucose subunits and β-glucans include one or more β-linkages between glucose subunits.

In various embodiments of the present invention, a glucan is linked or conjugated to a binding moiety as a component of a larger linked or conjugated molecule. A glucan may be linked or conjugated to a binding moiety as a component of a molecule including a plurality of glucans, e.g., a plurality of β-1,6-glucans or a plurality of assorted glucans, optionally including no or one or more β-1,6-glucans. A glucan may be linked or conjugated to a binding moiety as a component of a molecule including one or more glucans and one or more non-glucan components. Accordingly, the present invention encompasses not only binding moieties linked or conjugated to molecules including only glucan(s), but also binding moieties linked or conjugated to molecules including at least one glucan and one or more atoms, groups, molecules, or other biological or chemical entities other than a glucan.

β-1,6-glucans occur frequently in fungi but are rarer outside fungi. The glucan used in accordance with the invention can include β-1,6 glucan. In some embodiments, β-glucans are derived from Umbilicariaceae, such as U. pustulata and U. hirsute, U. angulata, U. caroliniana, and U. polyphylla.

In some embodiments, β-glucans are derived from Candida, such as C. albicans. Other organisms from which β-glucans may be used include Coccidioides immitis, Trichophyton verrucosum, Blastomyces dermatidis, Cryptococcus neoformans, Histoplasma capsulatum, Saccharomyces cerevisiae, Paracoccidioides brasiliensis, and Pythiumn insidiosum. In some embodiments, β-glucans are chemically or enzymatically synthesized, as is known in the art, or in other embodiments, β-glucans are derived from any species producing the same, and chemically or enzymatically altered, for example, to increase solubility or increase O-acetylation of the molecule.

In some embodiments, β-glucans are fungal glucans. A “fungal” glucan will generally be obtained from a fungus but, where a particular glucan structure is found in both fungi and non-fungi (e.g., in bacteria, lower plants or algae) then the non-fungal organism may be used as an alternative source. In some embodiments, β-glucans are made genetically.

Full-length native β-glucans are insoluble and have a molecular weight in the megadalton range. In some embodiments, this invention provides soluble β-1,6-glucan. In some embodiments, this invention provides soluble O-acetylated β-1,6-glucan. In some embodiments, solubilization may be achieved by fragmenting long insoluble glucans. This may be achieved by, for example, hydrolysis or, in some embodiments, by digestion with a glucanase (e.g., with a β-1,3 glucanase or limited digestion with a β-1,6 glucanase). In other embodiments, glucans can be prepared synthetically, for example, and in some embodiments, by joining monosaccharide or disaccharide building blocks. O-acetylation of such glucans can readily be accomplished by methods known in the art. Such methods may include chemical and/or enzymatic acetylation, such as are known in the art.

There are various sources of fungal β-glucans. For instance, pure β-glucans are commercially available, e.g., pustulan (Calbiochem) is a β-1,6-glucan purified from Umbilicaria papullosa. β-glucans can be purified from fungal cell walls in various ways, for example, as described in Tokunaka et al. [(1999) Carbohydr. Res. 316:161-172], and the product may be enriched for β-1,6-glucan moieties, or O-acetylated β-1,6-glucan moieties, by methods as are known in the art.

One of ordinary skill in the art will be able to identify or select appropriate methods to enrich for β-1,6-glucan moieties and/or for O-acetylated β-1,6-glucan. In one embodiment, O-acetylation of β-glucan is performed chemically. For example, polysaccharides (50 mg) are dried in a speed vac centrifuge and resuspended in 1.5 mL of acetic anhydride (Mallindcrockdt). After resuspension, a few crystals of 4-dimethylaminopyridine (Avocado Research Chemist, Ltd) are added as catalyst. The reaction is allowed to proceed at room temperature for 5, 20, or 120 minutes and then stopped with 2 volumes of water. Afterwards the samples are dialyzed overnight against water. It will be appreciated that this process could be varied or scaled up, as evident to one of skill in the art. In other embodiments, methods for separating O-acetylated β-1,6-glucan include one or more of the following steps, which could be performed in various orders: (a) separation based on higher hydrophobicity, such as binding to any hydrophobic matrix/resin; (b) separation based on digestion with a suitable endo- or exo-glucanase or combination thereof, wherein the O-acetylated β-1,6-glucan is resistant to digestion; (c) affinity separation using antibodies or other moieties that bind to β-1,6-glucan or to β-acetyl groups thereon; (d) separation based on molecular weight. In one embodiment, β-1,6-glucan is digested with an enzyme that digests unacetylated and/or lightly acetylated β-1,6-glucan. The resulting material is separated based on size or molecular weight and a portion comprising heavily acetylated glucan is isolated. In some embodiments, β-1,6-glucan preparations are obtained, digested and O-acetylated oligosaccharides are separated or in another embodiment, isolated, and used in the preparation of new compositions. Such compositions represent embodiments of the β-1,6-glucan preparations enriched for O-acetylated residues of this invention.

It is to be understood that the products of any process for preparing enriched β-acetylated β-1,6-glucan preparations are to be considered as appropriate for use in methods and compositions of the present invention.

In some embodiments, glucans for use in compositions and methods of the invention may comprise structural modifications, e.g., structural modifications not present in native glucan preparations. Such modifications may comprise, e.g., O-acetylation, as described herein. In other embodiments, such modifications may comprise one or more of methylation, alkylation, alkoylation, sulfation, phosphorylation, lipid conjugation or other modifications, as are known to one skilled in the art. In some embodiments the modification comprises modification (e.g., esterification) with an acid such as formic, succinic, citric acid, or other acid known in the art.

In some embodiments, lipid conjugation to any or all free hydroxyl groups may be accomplished by any number of means known in the art, for example, as described in Drouillat et al. [(1998) Pharm. Sci. 87(1):25-30], Mbadugha et al. [(2003) Org. Lett. 5 (22), 4041-4044].

In some embodiments, methylation may be accomplished and verified by any number of means known in the art, for example, as described in Mischnick et al. [(1994) Carbohydr. Res. 264, 293-304]; Bowie et al. [(1984) Carbohydr. Res. 125, 301-307]; Sherman and Gray [(1992) Carbohydr. Res. 231, 221-235]; Stankowski and Zeller [(1992) Carbohydr. Res. 234, 337-341]; Harris et al. [(1984) Carbohydr. Res. 127, 59-73]; Carpitaand Shea [(1989) Linkage structure of carbohydrates by gas chromatography-mass spectrometry (GC-MS) of partially methylated alditol acetates. In Analysis of Carbohydrates by GLC and MS (Biermann, C J. & McGinnis, G. D., eds), pp. 157-216. CRC Press, Boca Raton, Fla.].

In some embodiments, methylation can be confirmed by GLC of further-derived TMS ethers, acetates or other esters, coupled MS, or digestion to monosaccharides, de-O-methylation and analysis by derivatization and GLC/MS, for example as described in Pazur [(1986) Carbohydrate Analysis—A Practical Approach, IRL Press, Oxford, pp. 55-96]; Montreuil et al. [(1986) Glycoproteins. In M. F. Chaplin and J. F. Kennedy, (eds.), Carbohydrate Analysis—a Practical Approach, IRL Press, Oxford, pp. 143-204]; Sellers et al. [(1990) Carbohydr. Res., 207, C1-C5]; O'Neill et al. [(1990) Pectic polysaccharides of primary cell walls. In P. M. Dey (ed.), Methods in Plant Biochemistry, Volume 2, Carbohydrates, Academic Press, London, pp. 415-441]; Stephen et al. [(1990) Methods in Plant Biochemistry, Volume 2, Carbohydrates, Academic Press, London, pp. 483-522]; or Churms [(1991) CRC Handbook of Chromatography. Carbohydrates, Volume II, CRC Press, Boca Raton, Fla., USA].

In some embodiments, phosphorylation, optionally including the introduction of other modifications, and verification of the obtained product may be accomplished by means well known to those skilled in the art, see for example, Brown [(1951) Biochem. Biophys. Acta 7, 487]; Roseman and Daffner [(1956) Anal. Chem. 28, 1743]; Romberg and Horecker [(1955) in Methods in enzymology, Vol. I, Academic Press, New York p. 323]; and U.S. Pat. No. 4,818,752.

In some embodiments, glucan sulfation and verification of the obtained product may be accomplished by any of the means well known in the art, for example, as described in Alban and Franz [(2001) Biomacromolecules 2, 354-361]; Alban et al. [(1992) Arzneimittelforschung 42, 1005-1008]; or Alban et al. [(2001) Carbohydr. Polym. 47, 267-276].

In various embodiments, β-1-6 glucan of the present invention includes β-1-6 glucan enriched for O-acetylated groups. In one embodiment, in any of the preparations for use according to the methods of the invention, the glucan contains at least 25% by weight O-acetylated glucan. In one embodiment, in any of the preparations for use according to the methods of the invention, the glucan contains from about 15% to about 30% by weight O-acetylated glucan. In another embodiment, in any of the preparations for use according to the methods of the invention, the glucan contains from about 10% to about 35% by weight O-acetylated glucan, or in another embodiment, from about 20% to about 50% by weight O-acetylated glucan, or in another embodiment, from about 25% to about 60% by weight O-acetylated glucan, or in another embodiment, from about 35% to about 80% by weight O-acetylated glucan, or in another embodiment, from about 18% to about 35% by weight O-acetylated glucan, or in another embodiment, from about 15% to about 75% by weight O-acetylated glucan. In other embodiments, the glucan contains between about 75% and 100% by weight O-acetylated glucan, e.g., between 75% and 90%, or between 90% and 100% by weight O-acetylated glucan. In one embodiment, in any of the preparations for use according to the methods of the invention, the glucan contains approximately that percentage of O-acetylated glucose units (by weight or number, in various embodiments of the invention) that would result from digestion of a naturally occurring β-1-6 glucan (e.g., pustulan or any other β-1-6 glucan mentioned herein) with a β-1-6 endoglucanase for a time sufficient to digest at least 90% by weight of the β-1-6 glucan to oligosaccharides comprising 5 or fewer glucose units followed by (i) removal of those oligosaccharides comprising 5 or fewer glucose residues from the composition or (ii) isolation of a portion of the resulting composition having a molecular weight greater than 5 kD, or in some embodiment greater than 10, 20, 30, 50, or 100 kD. In some embodiments, the term “enriched for O-acetylated residues” refers to the enhanced % of O-acetylated sites in individual glucose units within the glucan molecule, enhanced % of O-acetylated glucose units within the glucan molecule, or a combination thereof, as compared to a native glucan molecule. In one embodiment, reference to glucan preparations enriched by a particular weight percent for O-acetylated glucan, refers to preparations comprising an enhanced % of O-acetylated sites in individual glucose units within the glucan molecule, an enhanced % of O-acetylated glucose units within the glucan molecule, or a combination thereof, as compared to a glucan molecule.

Glucans derived from different sources may comprise varying amounts of O-acetylation in terms of O-acetylated sites in individual glucose units, O-acetylated glucose units within the glucan molecule, or a combination thereof. According to this aspect of the invention, the term “enriched for O-acetylated glucan” refers, in some embodiments, to enhanced O-acetylation as described herein, between the reference source from which the glucan is derived, and may not represent an overall enrichment as compared to any glucan source.

In one embodiment, the term “enriched for O-acetylated glucan” refers, to an enrichment of at least 25% by weight of the glucan chains, which are O-acetylated on at least one glucose unit, or at least 25% of the glucose units present in the glucan in the composition are O-acetylated, or a combination thereof. In some embodiments, at least 25% of the glucose units in at least 1%, or in another embodiment, at least 5% of the β-glucan chains are O-acetylated. In other embodiments between 25% and 35%, between 25% and 50%, between 25% and 75%, between 15% and 45%, between 20% and 60%, between 35% and 80%, or others of the glucose units in at least 5% of the β-glucan chains are O-acetylated, etc. In other embodiments, embodiments between 25% and 35%, between 25% and 50%, between 25% and 75%, between 15% and 45%, between 20% and 60%, between 35% and 80%, or others of the glucose units, in at least 10% of the β-glucan chains, or in another embodiment, in at least 15% of the β-glucan chains, or in another embodiment, in at least 20% of the β-glucan chains, are O-acetylated.

In various embodiments of the present invention, glucans of the present invention, e.g., β-1-6 glucan of the present invention, includes low molecular weight glucans. In various embodiments of the present invention, glucans of the present invention, e.g., β-1-6 glucan of the present invention, includes glucans having a molecular weight of less than 100 kDa (e.g., less than 80, 70, 60, 50, 40, 30, 25, 20, or 15 kDa).

In various embodiments of the present invention, a glucan of the present invention, e.g., β-1-6 glucan of the present invention, includes an oligosaccharide e.g., an oligosaccharide containing 85 or fewer (e.g., 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, or 3) glucose monosaccharide units.

In some embodiments β-1,6-glucan used in the methods and compositions of the present invention include a low molecular weight glucan. In some embodiments of any method of the invention in which β-1,6-glucan is utilized, the β-1,6-glucan comprises or consists essentially of a low molecular weight glucan. Optionally, in certain embodiments, at least some of the low molecular weight β-1,6-glucan in any embodiment of the invention is enriched for O-acetylated groups.

In certain embodiments of the invention at least 10%, 20%, 30%, 40%, 50%, 60%, 70%, 80%, 90%, 95%, 98%, or 99% of the glucan contained in the composition by weight is β-1,6 glucan. In certain embodiments between 20% and 50% of the glucan contained in the composition is β-1,6 glucan. In certain embodiments between 50% and 100% of the glucan contained in the composition is β-1,6 glucan. In one embodiment of any of the compositions or methods of the invention, the glucan contains from about 15% to about 30% by weight β-1,6 glucan. In another embodiment of any of the compositions or methods of the invention, the glucan contains from about 10% to about 35% by weight β-1,6 glucan, or in another embodiment, from about 20% to about 50% by weight β-1,6 glucan, or in another embodiment, from about 25% to about 60% by weight β-1,6 glucan, or in another embodiment, from about 35% to about 80% by weight β-1,6 glucan, or in another embodiment, from about 18% to about 35% by weight β-1,6 glucan, or in another embodiment, from about 15% to about 75% by weight β-1,6 glucan. In certain embodiments, said glucan is a mixture of oligomers or polymers, wherein the β-1,6 glucan is greater than about 10%, 20%, 30%, 40%, 50%, 60%, 70%, 80%, 90%, 95%, 98%, or 99% by weight of those oligomers or polymers. In certain embodiments the composition contains less than 50%, 40%, 30%, 20%, 10%, or 5% β-1,3 glucan by weight. In certain embodiments of the invention “weight” refers to “dry weight”.

A glucan of the present invention may be associated with a binding moiety of the present invention by any means known in the art. For instance, the glucan and binding moiety may be covalently bound, directly bound, indirectly bound, or bound via a linker. In various embodiments of the present invention, the number or type of glucans present in a glucan-linked binding moiety may vary. In various embodiments of the present invention, including embodiments encompassed herein including any number or type of glucan molecules, a glucan-linked binding moiety may include a single point of direct or indirect linkage of the binding moiety to one or more glucan molecules, or may include a plurality of distinct direct or indirect points of linkage with one or more glucan molecules. For instance, a binding moiety may be conjugated to one or more glucan molecules via one or more distinct atoms of the binding moiety or may be conjugated to a linker conjugated to one or more glucan molecules via one or more distinct atoms of the binding moiety. Various methods of conjugation or other means of associating a first molecule with a second molecule, e.g., one or more glucans with a binding moiety, are known in the art, including a variety of means of utilizing various linkers known in the art to associate one or more of a first molecule with one or more of a second molecule, e.g., one or more glucans with a binding moiety.

Linking a binding moiety to a glucan of the present invention may be accomplished, e.g., by any means known in the art, e.g., as described in U.S. Pat. No. 5,965,714, or United States Patent Publication No. 20070141084, or Schneerson et al. [(2003) Proc. Natl. Acad. Sci. USA. 100 (15):8945-50], Lees et al. [(1996) Vaccine 14 (3):190-8], or via the use of a cross-linking agent as described herein, or other methods, as will be appreciated by one skilled in the art.

In some embodiments, glycosylated antibodies are used and β-1,6-glucan is linked to a glycosylated residue of an antibody, or in some embodiments, linkages may be multiple and involve multiple sites on the antibody, or binding moiety, as will be understood by one skilled in the art.

In some embodiments, linking a glucan to a targeting moiety may result in enhanced phagocytosis and/or killing of the targeted cell or organism. In some embodiments, such lysis may be mediated by any professional antigen presenting cell or killer cell, such as, for example, neutrophils, macrophages, dendritic cells, natural killer cells, cytotoxic T lymphocytes, and others.

Various embodiments of the present invention do not exclude that a glucan-conjugated binding moiety may be further associated with one or more atoms or molecules that are neither a component of the glucan nor a component of the binding moiety. Accordingly, the present invention encompasses molecules comprising a glucan, a binding moiety, and, additionally, any of one or more atoms, one or more compounds, one or more molecules, one or more amino acids, one or more proteins, one or more protein complexes, or the like which may be associated directly or indirectly with one or more of a glucan and a binding moiety by any means described herein or otherwise known in the art.

Particular embodiments of the present invention include, among other things, β-1,6-glucan directly linked to a binding moiety, e.g., an antibody capable of antagonizing PD-1; β-1,6-glucan directly linked to a binding moiety, e.g., an antibody capable of antagonizing CTLA-4; β-1,6-glucan directly linked to a binding moiety, e.g., an antibody capable of antagonizing TIM-3; β-1,6-glucan directly linked to a binding moiety, e.g., an antibody capable of antagonizing LAG-3; β-1,6-glucan directly linked to a binding moiety, e.g., an antibody capable of antagonizing ICOS; β-1,6-glucan directly linked to a binding moiety, e.g., an antibody capable of antagonizing TIGIT; and β-1,6-glucan directly linked to a binding moiety, e.g., an antibody capable of agonizing CD40.

Particular embodiments of the present invention include, among other things, β-1,6-glucan indirectly linked to a binding moiety, e.g., an antibody capable of antagonizing PD-1; β-1,6-glucan indirectly linked to a binding moiety, e.g., an antibody capable of antagonizing CTLA-4; β-1,6-glucan indirectly linked to a binding moiety, e.g., an antibody capable of antagonizing TIM-3; β-1,6-glucan indirectly linked to a binding moiety, e.g., an antibody capable of antagonizing LAG-3; β-1,6-glucan indirectly linked to a binding moiety, e.g., an antibody capable of antagonizing ICOS; β-1,6-glucan indirectly linked to a binding moiety, e.g., an antibody capable of antagonizing TIGIT; and β-1,6-glucan indirectly linked to a binding moiety, e.g., an antibody capable of agonizing CD40.

Particular embodiments of the present invention include, among other things, β-1,6-glucan directly linked to pembrolizumab, β-1,6-glucan directly linked to nivolumab, β-1,6-glucan directly linked to the anti-CTLA-4 antibody ipilumumab, and β-1,6-glucan directly linked to the anti-GITR antibody MK-4166.

Particular embodiments of the present invention include, among other things, β-1,6-glucan indirectly linked to pembrolizumab, β-1,6-glucan indirectly linked to nivolumab, β-1,6-glucan indirectly linked to the anti-CTLA-4 antibody ipilumumab, and β-1,6-glucan indirectly linked to the anti-GITR antibody MK-4166.

### Combination Therapies

The present invention includes, in various embodiments, a β-1,6-glucan conjugated to a binding moiety directed to a surface feature of a T cell, a T regulatory cell, or a Myeloid Derived Suppressor Cell, such that the binding results in the cumulative effect of increasing immune activity, e.g., tumor-targeted immune activity and/or T cell immune activity, e.g., tumor-targeted T cell immune activity.

In various embodiments of the present invention, a β-1,6-glucan-conjugated binding moiety may be administered to a subject in need thereof in combination with one or more additional agents which may be administered at one or more different times relative to the β-1,6-glucan-conjugated binding moiety.

In various embodiments, the additional agent is a second, separate binding moiety optionally directly or indirectly linked to a β-1,6-glucan. For instance, the additional agent is a β-1,6-glucan-linked binding moiety directed to a tumor or cancer cell, e.g., via a surface feature of a tumor or cancer cell. In particular embodiments, the additional agent includes a binding moiety directed to a tumor or cancer cell present in a subject to which the additional agent is administered.

In various embodiments of the present invention, the additional agent is a cancer therapeutic, e.g., a cancer therapeutic known in the art, e.g., a cancer therapeutic known in the art for the treatment a cancer or cancer cell present in a subject to which the second agent is administered. Various cancer therapeutics and various uses thereof are known in the art. Cancer therapeutics include, without limitation, a chemotherapeutic agent selected from the group consisting of a cisplatin, doxorubicin, gemcitabine, docetaxel, paclitexel, and belomycin. In certain embodiments, said antineoplastic agent is selected from the group consisting of spiroplatin, cisplatin, carboplatin, methotrexate, fluorouracil, adriamycin, mitomycin, ansamitocin, bleomycin, cytosine arabinoside, arabinosyl adenine, mercaptopolylysine, vincristine, busulfan, chlorambucil, melphalan, PAM, L-PAM, phenylalanine mustard, mercaptopurine, mitotane, procarbazine hydrochloride actinomycin D, daunorubicin hydrochloride, doxorubicin hydrochloride, paclitaxel and other taxenes, rapamycin, manumycin A, TNP-470, plicamycin, mithramycin, aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, interferon α2a, interferon α2b, teniposide (VM-26), vinblastine sulfate (VLB), vincristine sulfate, bleomycin sulfate, hydroxyurea procarbazine, and dacarbazine.

In various embodiments of the present invention, the additional agent is a source of anti-β-1,6-glucan antibodies. Exemplary sources of anti-β-1,6-glucan antibodies include purified anti-β-1,6-glucan antibodies or pooled human IgGs (e.g., 10% IVIG available from Baxter). In various embodiments the additional agent which is a source a β-1,6-glucan-conjugated binding moiety is administered to the subject prior to administration of a β-1,6-glucan-conjugated binding moiety. As used herein, the term “prior to” encompasses administration at any time prior to administration of a β-1,6-glucan-conjugated binding moiety (e.g., at least 1 minute, at least 5 minutes, at least 1 hour, at least 12 hours, at least 1 day, at least 2 days, at least 1 week, at least 1 month, etc.). In various embodiments the additional agent which is a source a β-1,6-glucan-conjugated binding moiety is administered to the subject concurrently with administration of a β-1,6-glucan-conjugated binding moiety (e.g., in the same composition or in a separate composition). As used herein, the term “concurrently” means at substantially the same time and encompasses, e.g., co-administration in the same composition and parallel administrations of two separate compositions which may be administered via the same or different route.

In various embodiments of the present invention, the additional agent is an agent (e.g., a source of β-1,6-glucan) that will promote the production of endogenous anti-β-1,6-glucan antibodies within the subject. Exemplary sources of β-1,6-glucan include any of sources described herein (e.g., pustulan and the like) and certain foods or dietary supplements (e.g., bread or any other food or dietary supplement which includes β-1,6-glucan). In various embodiments the additional agent which is an agent (e.g., a source of β-1,6-glucan) that will promote the production of endogenous anti-β-1,6-glucan antibodies is administered to the subject prior to administration of a β-1,6-glucan-conjugated binding moiety. In various embodiments the additional agent which is an agent (e.g., a source of β-1,6-glucan) that will promote the production of endogenous anti-β-1,6-glucan antibodies is administered to the subject concurrently with administration of a β-1,6-glucan-conjugated binding moiety.

In various embodiments of the present invention, the additional agent is an agent which biases antibody production to yield relatively greater amounts of immunoglobulin G (IgG) 1, 2 or 3 versus immunoglobulin G (IgG) 4. In various embodiments of the present invention, the additional agent is an agent which biases antibody production to yield relatively greater amounts of IgG₂ versus IgG₄. In various embodiments, the biasing agent is a cytokine, e.g., interleukin-2, interleukin-12 or interferon-γ or a combination thereof. In various embodiments, the biasing agent downmodulates interleukin-4 or interleukin-10 production or interferes with interleukin-4 or interleukin-10 activity. In various embodiments, the biasing agent is administered prior to or concurrently with a source of β-1,6-glucan. In various embodiments the biasing agent (and optionally the source of β-1,6-glucan) is administered to the subject prior to administration of a β-1,6-glucan-conjugated binding moiety. In various embodiments the biasing agent (and optionally the source of β-1,6-glucan) is administered to the subject concurrently with administration of a β-1,6-glucan-conjugated binding moiety.

### Applications

Methods and compositions of the present invention are useful in a variety of medical applications. In particular embodiments, methods and compositions of the present invention can be used in the treatment of a tumor or cancer, e.g., a cancer selected from Adrenal Cancer, Anal Cancer, Bile Duct Cancer, Bladder Cancer, Bone Cancer, Brain/CNS Tumors In Adults, Brain/CNS Tumors In Children, Breast Cancer, Breast Cancer In Men, Cancer in Adolescents, Cancer in Children, Cancer in Young Adults, Cancer of Unknown Primary, Castleman Disease, Cervical Cancer, Colon/Rectum Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Leukemia—Acute Lymphocytic (ALL) in Adults, Leukemia—Acute Myeloid (AML), Leukemia—Chronic Lymphocytic (CLL), Leukemia—Chronic Myeloid (CML), Leukemia—Chronic Myelomonocytic (CMML), Leukemia in Children, Liver Cancer, Lung Cancer, Lung Cancer—Non-Small Cell, Lung Cancer—Small Cell, Lung Carcinoid Tumor, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Non-Hodgkin Lymphoma In Children, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Sarcoma—Adult Soft Tissue Cancer, Skin Cancer, Skin Cancer—Basal and Squamous Cell, Skin Cancer—Melanoma, Skin Cancer—Merkel Cell, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor.

In various embodiments, methods and compositions of the present invention can be used to treat one or more tumors or cancers in a subject in which treatment not including a β-1,6-glucan (or not including a β-1,6-glucan linked to a binding moiety directed to a T regulatory cell) has been previously administered and has not been effective, and/or wherein the subject, tumor, or cancer is refractory to a prior treatment, e.g., treatment by one or more agents not including a β-1,6-glucan (or not including a β-1,6-glucan linked to a binding moiety directed to a T regulatory cell).

In various embodiments of the present invention, linkage to a β-1,6-glucan of the present invention provides a means of, or can be used in a method of, increasing the immune activity of a known binding moiety directed to a T regulatory cell or T cell.

In various embodiments of the present invention, a β-1,6-glucan-linked binding moiety is directed to a T regulatory cell mediates inhibition, or is capable of mediating inhibition, of the activity of a cell to which it binds.

In various embodiments of the present invention, a β-1,6-glucan-linked binding moiety is directed to a T regulatory cell kills, is capable of killing, mediates killing of, or is capable of mediating killing of, a cell to which it binds. In certain instances inhibition of a cell occurs by or includes killing the cell, e.g., a T regulatory cell, as the result of or mechanism of the inhibition or the treatment inducing the inhibition.

Without wishing to adhere to any particular scientific theory, the present invention also encompasses situations where the treatment of a tumor or cancer by a β-1,6-glucan-linked binding moiety as provided herein is mediated by an immunostimulatory effect of β-1,6-glucan localization independent of, or including an aspect independent of, the immediate or direct impact of the β-1,6-glucan-linked binding moiety on a cell to which the β-1,6-glucan-linked binding moiety binds. Indeed, without wishing to adhere to any particular scientific theory, the present invention stems in part from the recognition that (a) checkpoint molecules are expressed on cells that are in the tumor microenvironment, (b) checkpoint inhibitor antibodies will have their normal intended effect (e.g., modulating Tregs) and (c) the conjugated β-1,6-glucan will recruit neutrophils or other immune cells, which should in turn lead to depletion of Tregs and/or depletion of cancer or tumor cells.

In various embodiments of the present invention, a β-1,6-glucan-linked binding moiety provides a means of, or is used in a method of, modulating an immune response, e.g., against a tumor or cancer in a subject in need thereof.

In general, the compositions and agents described herein may be administered to a subject in any effective, convenient manner including, for instance, administration by intravascular (i.v.), intramuscular (i.m.), intranasal (i.n.), subcutaneous (s.c), oral, rectal, intravaginal delivery, or by any means in which the composition or agent can be delivered to the desired tissue or cells (e.g., to the tumor microenvironment). Suitable compositions and excipients are well known in the art and include those, e.g., that are used with pembrolizumab, nivolumab, ipilumumab, MK-4166 or other commercial antibodies. For example, compositions suitable for parenteral administration include aqueous and non-aqueous sterile injection solutions which may contain antioxidants, buffers, bacteriostats and solutes which render the composition isotonic with the blood of the intended recipient; and aqueous and nonaqueous sterile suspensions which may include suspending agents and thickening agents. The compositions may be presented in unit-dose or multi-dose containers, for example sealed ampules and vials, and may be stored in a freeze-dried (lyophilized) condition requiring only the addition of the sterile liquid excipient, for example water for injection, immediately prior to use. Unit dosage compositions are those containing a daily dose or unit daily sub-dose or an appropriate fraction thereof, of the therapeutic agent or agents. It will be understood that in addition to the ingredients particularly mentioned above, the compositions of this invention may include other agents or excipients conventional in the art having regard to the type of composition in question. It will be apparent to those skilled in the art that various modifications and variations can be made in the compositions, use and preparations of the present invention without departing from the spirit or scope of the invention.

### EXAMPLES

The present examples are included to illustrate at least one of the various embodiments of the present invention in which a binding moiety is directed to a T regulatory cell is linked to a β-1,6-glucan. The below examples are not limiting to any of the embodiments provided herein or to the scope of the present invention.

### Example 1

### β-1,6-Glucan Conjugated to an Anti-CTLA-4 Monoclonal Antibody is Functional

The anti-CTLA-4 antibody 9D9 (mouse anti-mouse CTLA-4; BioXCell) was conjugated to β-1,6-glucan. Conjugates were characterized for average sugar load by MALDI-TOF, and assayed for functionality by binding polyclonal anti-β-1,6-glucan present in human serum.

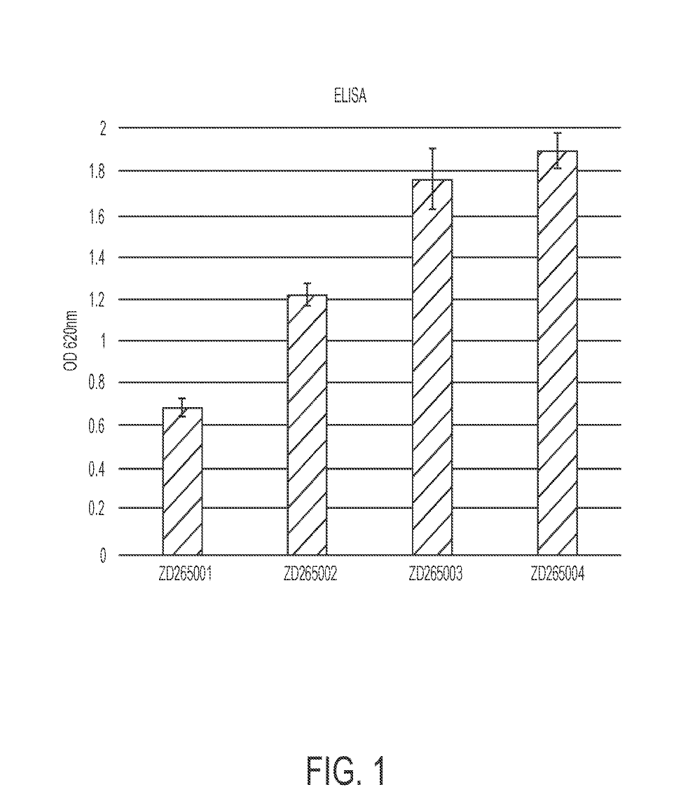

In an ELISA assay (FIG. 1), the extracellular domain of CTLA-4 (Sino Biological) was adsorbed to 96-well plates. Following blocking, naked anti-CTLA-4 antibody 9D9 or conjugates were added to the plate at various concentrations. The antibodies were reacted with polyclonal human anti-β-1,6-glucan antibodies, which were detected by fluorescently labeled anti-human secondary antibodies (FIG. 1). FIG. 1 includes four columns showing data from conjugates having different sugar loads and different levels of binding by anti-β-1,6-glucan antibodies. FIG. 1 shows increased binding of anti-β-1,6-glucan antibodies for the conjugates. The average molecular weight of the antibodies were determined by MALDI-TOF. The functionality of the conjugates as assayed by the ability to bind naturally occurring anti-β-1,6-glucan antibodies correlated with the load.

### Example 2

### Conjugation of β-1,6-Glucan Oligosaccharides to Monoclonal Cancer-Targeted Antibodies Improves Efficacy

The present Example demonstrates that conjugation of an antibody, e.g., a checkpoint inhibitor, to β-1,6-glucan improves the efficacy of that antibody. The present Example demonstrates that efficacy of an antibody conjugated to β-1,6-glucan may be further increased in the presence of or by administration of anti-β-1,6-glucan antibodies and/or by immunizing for β-1,6-glucan.

Without wishing to be bound by any particular scientific theory, data of the present Example supports the scientific theory that upon binding of an antibody conjugated to β-1,6-glucan to targets of the antibody, e.g., at high density, the β-1,6-glucan oligosaccharides are bound by endogenous anti-β-1,6-glucan antibodies, which endogenous anti-β-1,6-glucan antibodies in turn can activate the complement system, and then become targets for neutrophils.

In the present Example, an anti-CTLA-4 antibody is conjugated to β-1,6-glucan. CTLA-4 is expressed on various T cells, including T regulatory cells, which are known to suppress the immune response to tumors. Known anti-human CTLA4 antibodies include ipilimumab, which is approved for metastatic melanoma. The conjugated anti-CTLA-4 antibody of the present example is 9D9, a mouse IgG2b antibody similar to ipilimumab.

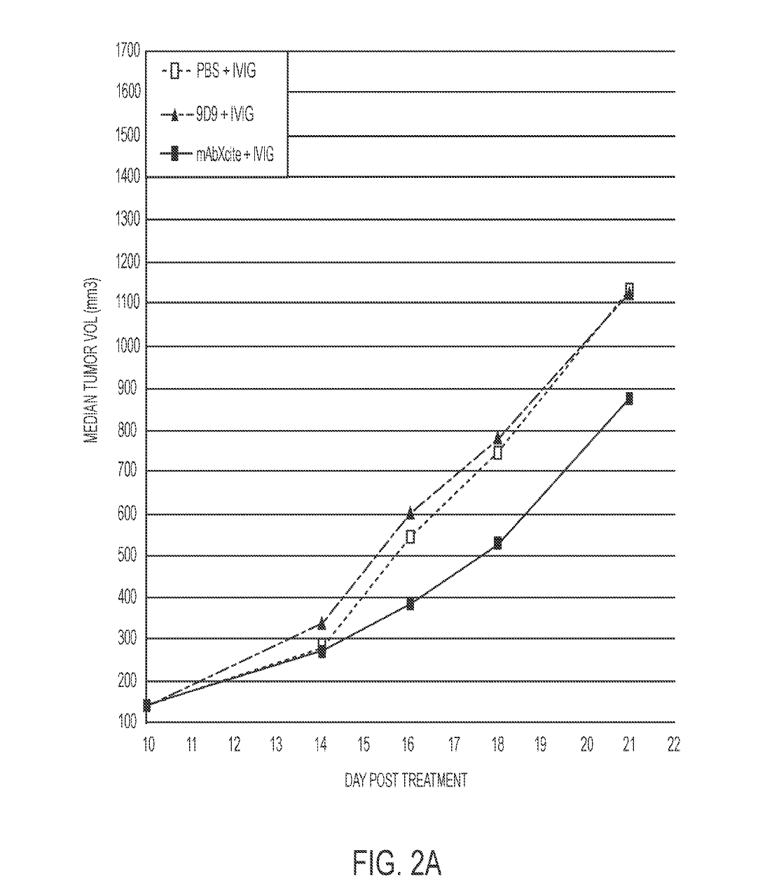

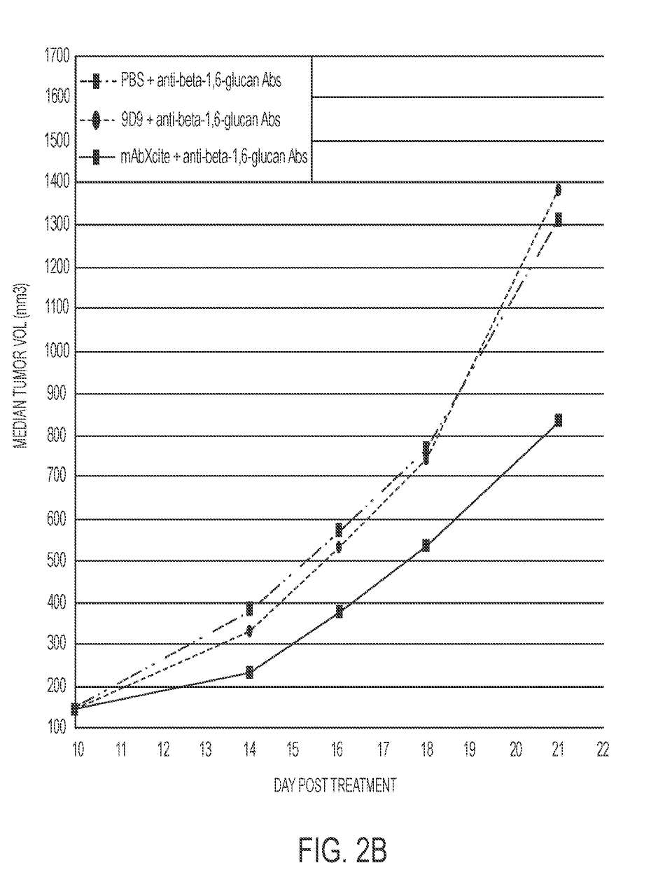

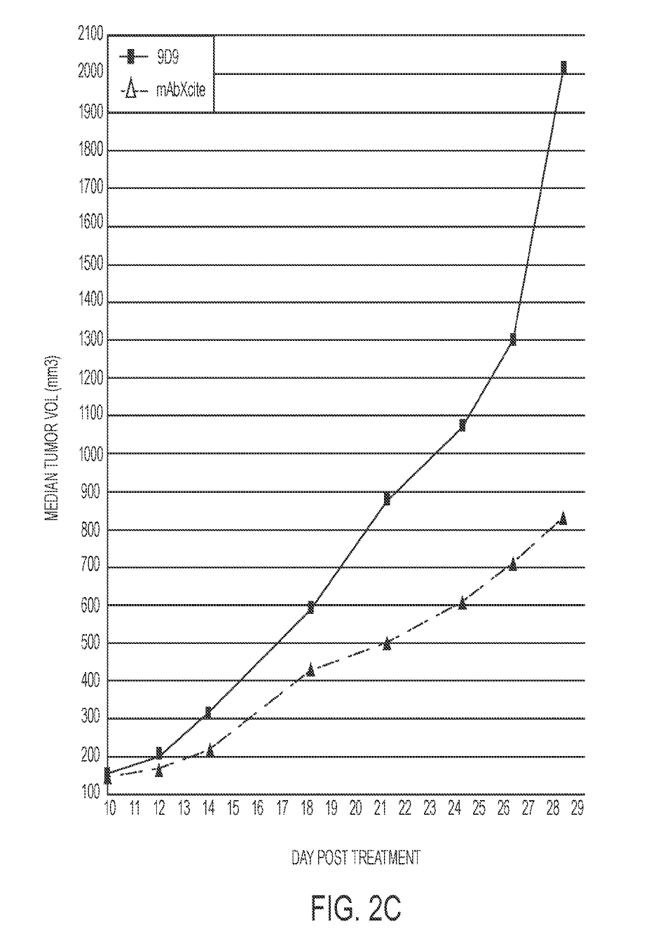

In a first experiment, anti-CTLA-4 antibody conjugated to β-1,6-glucan and unconjugated anti-CTLA-4 antibody were administered to BALB/c mice that had been implanted subcutaneously with 10⁶ CT-26 tumor cells. Certain of these mice were further administered pooled human IgGs (100 μl of 10% IVIG, Baxter). Results demonstrated that efficacy of anti-CTLA-4 antibody conjugated to β-1,6-glucan was enhanced in BALB/c mice that received the pooled human IgGs (as measured by tumor volume; FIG. 2A).

In a further experiment, anti-CTLA-4 antibody conjugated to β-1,6-glucan and unconjugated anti-CTLA-4 antibody were administered to BALB/c mice (implanted subcutaneously with 10⁶ CT-26 tumor cells) in the presence of purified human anti-β-1,6-glucan antibodies (30 μg purified anti-β-1,6-glucan IgGs). It was considered that the purified human anti-β-1,6-glucan antibodies, which, as human antibodies, are immunogenic in mice, would be even more immunogenic in the presence of the anti-CTLA-4 antibody, which could lead to clearance of the anti-β-1,6-glucan antibodies. A decreased dosage of human IgGs (30 μg purified anti-β-1,6-glucan antibodies vs. 10 mg IVIG) had lower immunogenicity (this preparation is still immunogenic but the dose is lower) and enhanced efficacy of anti-CTLA-4 antibodies conjugated to β-1,6-glucan (FIG. 2B).

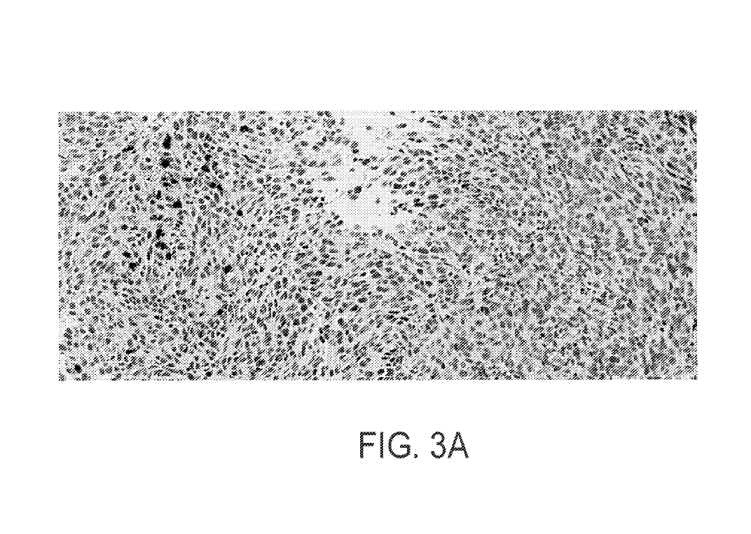

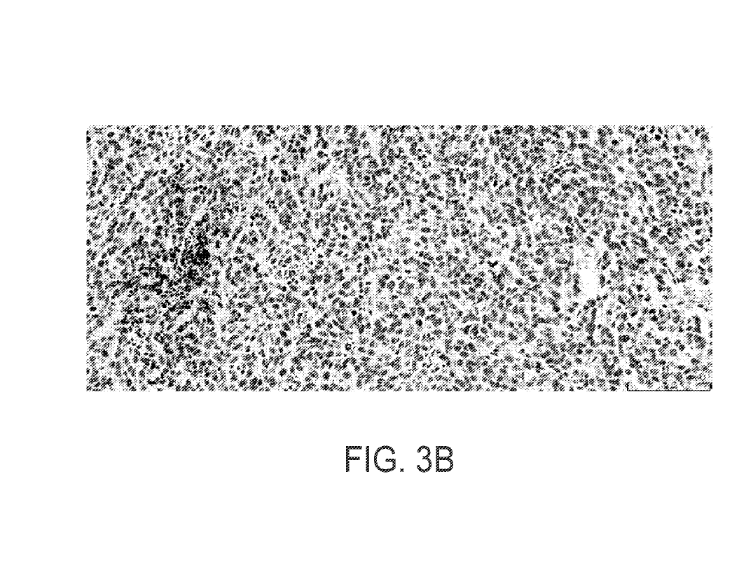

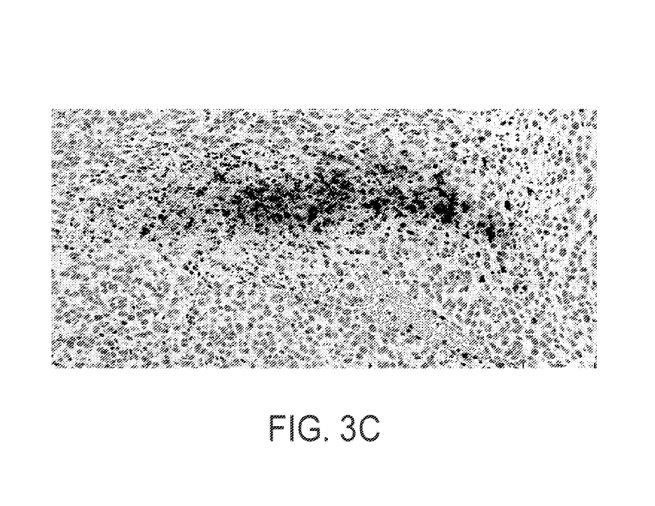

In a further experiment, a method of immunizing mice for β-1,6-glucan by supplementing their regular diet with bread was tested. Anti-CTLA-4 antibody conjugated to β-1,6-glucan and unconjugated anti-CTLA-4 antibody were administered to BALB/c mice (implanted subcutaneously with 10⁶ CT-26 tumor cells) fed with bread supplement. Mice fed a diet supplemented with bread showed increased efficacy of the anti-CTLA-4 antibody conjugated to β-1,6-glucan (as measured by tumor volume; FIG. 2C), which is associated with massive infiltration of neutrophils (FIG. 3).

Data of the present Example broadly relate to the modification of antibodies having targets expressed on T cells and/or of checkpoint inhibitors. While CTLA-4 is discussed in particular, at least one similar target on T cells is PD-1; PD-1 is the target for two approved antibodies, pembrolizumab and nivolumab, which are currently approved for metastatic melanoma (both antibodies) and non-small cell lung cancer (pembrolizumab). These antibodies, as well as others in development, often referred to as checkpoint inhibitors, “release the breaks” from T cells, that typically prevent recognition of self-antigens on normal cells, and in doing so facilitate recognition of cancer cells.

The present Example demonstrates that antibody targeting CTLA-4 conjugated to β-1,6-glucan is more efficacious than the unconjugated antibody. The present Example demonstrates that efficacy correlates with the anti-β-1,6-glucan antibodies available to the mice and with neutrophil infiltration. The level of efficacy observed is associated with anti-β-1,6-glucan antibodies and their availability, so that mouse anti-β-1,6-glucan antibodies mediate greater efficacy than human antibodies that are immunogenic in mice and can be cleared.

Taken together, the data suggest that the efficacy of checkpoint inhibitors, e.g., in a clinical setting, will be increased by conjugation to β-1,6-glucan, and may rely on pre-existing anti-β-1,6-glucan antibodies, or may be further increased by administration of anti-β-1,6-glucan antibodies and/or by immunizing for β-1,6-glucan.

### Other Embodiments

While we have described a number of embodiments of this invention, it is apparent that our basic disclosure and examples may be altered to provide other embodiments that utilize the compositions and methods of this invention. Therefore, it will be appreciated that the scope of this invention is to be defined by the appended claims rather than by the specific embodiments that have been represented by way of example. All references cited herein are hereby incorporated by reference.

What is claimed is:

1. A composition comprising β-1,6-glucan linked to an antibody, wherein the antibody is directed to a surface feature of a T regulatory cell.

2. The composition of claim 1, wherein the antibody is a monoclonal antibody.

3. The composition of claim 1, wherein the antibody is a human antibody.

4. The composition of claim 1, wherein the antibody is a humanized antibody.

5. The composition of claim 1, wherein the antibody is an antibody fragment.

6. The composition of claim 1, wherein the β-1,6-glucan is derived from a fungal glucan.

7. The composition of claim 1, wherein the β-1,6-glucan is derived from pustulan.

8. The composition of claim 1, wherein the β-1,6-glucan is derived from a fungal glucan via one or more processing steps that include hydrolysis and/or enzymatic digestion.

9. The composition of claim 1, wherein the β-1,6-glucan is chemically synthesized.

10. The composition of claim 1, wherein the β-1,6-glucan is genetically engineered.

11. The composition of claim 1, wherein the β-1,6-glucan is synthesized in a bacteria, yeast, or mammalian cell.

12. The composition of claim 1, wherein the β-1,6-glucan is soluble.

13. The composition of claim 1, wherein the β-1,6-glucan comprises a structural modification selected from the group consisting of O-acetylation, methylation, alkylation, esterification, alkoylation, sulfation, phosphorylation, lipid conjugation and combinations thereof.

14. The composition of claim 1, wherein the β-1,6-glucan has a molecular weight less than 100 kDa.

15. The composition of claim 1, wherein the β-1,6-glucan contains fewer than 85 glucose monosaccharide units.

16. The composition of claim 1, wherein the β-1,6-glucan contains 85, 84, 83, 82, 81, 80, 79, 78, 77, 76, 75, 74, 73, 72, 71, 70, 69, 68, 67, 66, or 65 glucose monosaccharide units.

17. The composition of claim 1, wherein the β-1,6-glucan contains 64, 63, 62, 61, 60, 59, 58, 57, 56, 55, 54, 53, 52, 51, 50, 49, 48, 47, 46, 45, or 44 glucose monosaccharide units.

18. The composition of claim 1, wherein the β-1,6-glucan contains 43, 42, 41, 40, 39, 38, 37, 36, 35, 34, 33, 32, 31, 30, 29, 28, 27, 26, 25, 24, or 23 glucose monosaccharide units.

19. The composition of claim 1, wherein the β-1,6-glucan contains 22, 21, 20, 19, 18, 17, 16, 15, 14, 13, 12, 11, 10, 9, 8, 7, 6, 5, 4, or 3 glucose monosaccharide units.

20. The composition of claim 1, wherein at least 90% of the dry weight of glucan contained in the composition is β-1,6-glucan.

21. The composition of claim 1, wherein less than 10% of the dry weight of glucan contained in the composition is β-1-3-glucan.

22. The composition of claim 1, wherein the composition is substantially free of β-1-3-glucan.

23. The composition of claim 1, wherein the β-1,6-glucan is directly linked to the antibody.

24. The composition of claim 1, wherein the β-1,6-glucan is linked to the antibody via a linker molecule.

25. The composition of claim 1, wherein the β-1,6-glucan is linked to the antibody via amide, aldehyde, urethane, imine or disulfide formation between the respective molecules, or between a linker molecule and the respective molecules.

26. A method of treating cancer, said method comprising administering to a subject in need thereof a composition comprising β-1,6-glucan linked to an antibody, wherein the antibody is directed to a surface feature of a T regulatory cell.

27. The method of claim 26, wherein the antibody is a monoclonal antibody.

28. The method of claim 26, wherein the antibody is a human antibody.

29. The method of claim 26, wherein the antibody is a humanized antibody.

30. The method of claim 26, wherein the antibody is an antibody fragment.

31. The method of claim 26, wherein the β-1,6-glucan is derived from a fungal glucan.

32. The method of claim 26, wherein the β-1,6-glucan is derived from pustulan.

33. The method of claim 26, wherein the β-1,6-glucan is derived from a fungal glucan via one or more processing steps that include hydrolysis and/or enzymatic digestion.

34. The method of claim 26, wherein the β-1,6-glucan is chemically synthesized.

35. The method of claim 26, wherein the β-1,6-glucan is genetically engineered.

36. The method of claim 26, wherein the β-1,6-glucan is synthesized in a bacteria, yeast, or mammalian cell.

37. The method of claim 26, wherein the β-1,6-glucan is soluble.

38. The method of claim 26, wherein the β-1,6-glucan comprises a structural modification selected from the group consisting of O-acetylation, methylation, alkylation, esterification, alkoylation, sulfation, phosphorylation, lipid conjugation and combinations thereof.

39. The method of claim 26, wherein the β-1,6-glucan has a molecular weight less than 100 kDa.

40. The method of claim 26, wherein the β-1,6-glucan contains fewer than 85 glucose monosaccharide units.

41. The method of claim 26, wherein the β-1,6-glucan contains 85, 84, 83, 82, 81, 80, 79, 78, 77, 76, 75, 74, 73, 72, 71, 70, 69, 68, 67, 66, or 65 glucose monosaccharide units.

42. The method of claim 26, wherein the β-1,6-glucan contains 64, 63, 62, 61, 60, 59, 58, 57, 56, 55, 54, 53, 52, 51, 50, 49, 48, 47, 46, 45, or 44 glucose monosaccharide units.

43. The method of claim 26, wherein the β-1,6-glucan contains 43, 42, 41, 40, 39, 38, 37, 36, 35, 34, 33, 32, 31, 30, 29, 28, 27, 26, 25, 24, or 23 glucose monosaccharide units.

44. The method of claim 26, wherein the β-1,6-glucan contains 22, 21, 20, 19, 18, 17, 16, 15, 14, 13, 12, 11, 10, 9, 8, 7, 6, 5, 4, or 3 glucose monosaccharide units.

45. The method of claim 26, wherein at least 90% of the dry weight of glucan contained in the composition is β-1,6-glucan.

46. The method of claim 26, wherein less than 10% of the dry weight of glucan contained in the composition is β-1-3-glucan.

47. The method of claim 26, wherein the composition is substantially free of β-1-3-glucan.

48. The method of claim 26, wherein the β-1,6-glucan is directly linked to antibody.

49. The method of claim 26, wherein the β-1,6-glucan is linked to the antibody via a linker molecule.

50. The method of claim 26, wherein the β-1,6-glucan is linked to the targeting moiety via amide, urethane, imine or disulfide formation between the respective molecules, or between a linker molecule and the respective molecules.

51. A method of modulating an immune response, said method comprising administering to a subject in need thereof a composition comprising β-1,6-glucan linked to an antibody, wherein the antibody is directed to a surface feature of a T regulatory cell.

52. The method of claim 51, wherein the antibody is a monoclonal antibody.

53. The method of claim 51, wherein the antibody is a human antibody.

54. The method of claim 51, wherein the antibody is a humanized antibody.

55. The method of claim 51, wherein the antibody is an antibody fragment.

56. The method of claim 51, wherein the β-1,6-glucan is derived from a fungal glucan.

57. The method of claim 51, wherein the β-1,6-glucan is derived from pustulan.

58. The method of claim 51, wherein the β-1,6-glucan is derived from a fungal glucan via one or more processing steps that include hydrolysis and/or enzymatic digestion.

59. The method of claim 51, wherein the β-1,6-glucan is chemically synthesized.

60. The method of claim 51, wherein the β-1,6-glucan is genetically engineered.

61. The method of claim 51, wherein the β-1,6-glucan is synthesized in a bacteria, yeast, or mammalian cell.

62. The method of claim 51, wherein the β-1,6-glucan is soluble.

63. The method of claim 51, wherein the β-1,6-glucan comprises a structural modification selected from the group consisting of O-acetylation, methylation, alkylation, esterification, alkoylation, sulfation, phosphorylation, lipid conjugation and combinations thereof.

64. The method of claim 51, wherein the β-1,6-glucan has a molecular weight less than 100 kDa.

65. The method of claim 51, wherein the β-1,6-glucan contains fewer than 85 glucose monosaccharide units.

66. The method of claim 51, wherein the β-1,6-glucan contains 85, 84, 83, 82, 81, 80, 79, 78, 77, 76, 75, 74, 73, 72, 71, 70, 69, 68, 67, 66, or 65 glucose monosaccharide units.

67. The method of claim 51, wherein the β-1,6-glucan contains 64, 63, 62, 61, 60, 59, 58, 57, 56, 55, 54, 53, 52, 51, 50, 49, 48, 47, 46, 45, or 44 glucose monosaccharide units.

68. The method of claim 51, wherein the β-1,6-glucan contains 43, 42, 41, 40, 39, 38, 37, 36, 35, 34, 33, 32, 31, 30, 29, 28, 27, 26, 25, 24, or 23 glucose monosaccharide units.

69. The method of claim 51, wherein the β-1,6-glucan contains 22, 21, 20, 19, 18, 17, 16, 15, 14, 13, 12, 11, 10, 9, 8, 7, 6, 5, 4, or 3 glucose monosaccharide units.

70. The method of claim 51, wherein at least 90% of the dry weight of glucan contained in the composition is β-1,6-glucan.

71. The method of claim 51, wherein less than 10% of the dry weight of glucan contained in the composition is β-1-3-glucan.

72. The method of claim 51, wherein the composition is substantially free of β-1-3-glucan.

73. The method of claim 51, wherein the β-1,6-glucan is directly linked to antibody.

74. The method of claim 51, wherein the β-1,6-glucan is linked to the antibody via a linker molecule.

75. The method of claim 51, wherein the β-1,6-glucan is linked to the antibody via amide, urethane, imine or disulfide formation between the respective molecules, or between a linker molecule and the respective molecules.

76. A composition comprising β-1,6-glucan linked to an antagonistic anti-PD1 antibody.

77. A composition comprising β-1,6-glucan linked to an antagonistic anti-CTLA-4 antibody.

78. A composition comprising β-1,6-glucan linked to an antagonistic anti-TIM-3 antibody.

79. A composition comprising β-1,6-glucan linked to an antagonistic anti-LAG-3 antibody.

80. A composition comprising β-1,6-glucan linked to an antagonistic anti-ICOS antibody.

81. A composition comprising β-1,6-glucan linked to an antagonistic anti-TIGIT antibody.

82. A composition comprising β-1,6-glucan linked to an agonistic anti-GITR antibody.

83. A composition comprising β-1,6-glucan linked to an agonistic anti-OX-40 antibody.

84. A composition comprising β-1,6-glucan linked to an agonistic anti-CD40 antibody.

85. A composition comprising β-1,6-glucan linked to pembrolizumab.

86. A composition comprising β-1,6-glucan linked to nivolumab.

87. A composition comprising β-1,6-glucan linked to ipilimumab.

88. A composition comprising β-1,6-glucan linked to MK-4166.
